(19) United States
(12) Patent Application Publication
(10) Pub. No.: US 2026/0165902 A1
(43) Pub. Date: Jun. 18, 2026

(54) PAD HAVING ELASTIC PORTIONS

(71) Applicant: Bauerfeind AG, Zeulenroda-Triebes (DE)

(72) Inventors: Heinrich Hess, Zeulenroda-Triebes (DE); Juliane Wiedner, Zeulenroda- Triebes (DE); Beatrix Bauerfeind-Johnson, Zeulenroda-Triebes (DE)

(21) Appl. No.: 19/124,834

(22) PCT Filed: Oct. 25, 2023
(86) PCT No.: PCT/EP2023/079764

§ 371 Date: Apr. 25, 2025

(30) Foreign Application Priority Data
Oct. 28, 2022 (DE) 10 2022 128 741.6

(51) Int. Cl. A61H 7/00; A61F 5/01

(52) U.S. Cl. CPC A61H 7/002; A61F 5/0109; A61F 5/0118; A61H 2201/1284; A61H 2201/1635; A61H 2201/164; A61H 2201/165; A61H 2201/1692; A61H 2205/06; A61H 2205/104

(57) ABSTRACT

The present invention relates to a pelotte for orthopaedic aids, comprising a pelotte base body with at least two expansion areas arranged in pairs.

The present invention relates to a pelotte for orthopedic aids, comprising a pelotte base body with at least two expansion areas arranged in pairs.

Furthermore, the invention relates to an orthopedic aid, in particular an orthosis or bandage, which has at least one such pelotte.

Pelottes used for various prophylactic and therapeutic applications are already known from the prior art, for example from the publications DE 27 22 563 C2, EP 0 598 291 A1 and EP 0 600 218 A2 .

Many patients with knee osteoarthritis suffer from retro-patellar osteoarthritis with pain when pressure is applied to the patella and then to the sliding bearing. Even moderate but persistent pressure can cause pain, for example when wearing tight jeans. The leading muscles around the large arthritic joints are often painfully hardened and shortened, and painful trigger point hardenings frequently occur on the muscle surface. In osteoarthritis of the knee, the tissue, the joint space, and the retropatellar area must be relaxed. Special targeted massage techniques are used for this in physiotherapy. In addition or as an alternative, bandages and orthoses with pelottes are used, which, through protrusions, as pelotte pressure elements such as nubs, produce a massage with release of the contractures and a sensorimotor response of the muscle when pressure is applied to the muscle. Pelottes with such protrusions, in particular nubs for massage, are not only known for the patella, but also for other areas of the base body and are described, for example, in DE 42 38 610 A1 and DE 10 2012 105 626 A1 .

The nubs act on the muscles, for example the “musculus vastus medialis,” in terms of sensorimotor function. When at rest, the pressure effect/compression is only caused by the tension of the knitted fabric of the bandage or orthosis. When the muscle is tensed, i.e., contracted, the pressure increases as a result of the increase in volume of the muscle, which generates a sensorimotor response that relieves pain. During movement, for example when walking due to knee and hip flexion, the nubs also cause an increase in pressure when weight is applied.

A disadvantage of state-of-the-art pelottes, such as patella pelottes, is that movement causes the pelotte to slip. For example, depending on the tensile resistance of the patellar pelotte, which is often made of silicone, the flexion of a patella pelotte can exert significant distal traction on the upper part of the bandage, which leads to a tendency to slip and, in arthritic patients, to uncomfortable pressure on the patella. At the same time, the distal pull reduces the vertically therapeutic pressure exerted by the nubs, partly because the nubs bend, thereby significantly weakening the desired sensorimotor response of the vastus medialis muscle.

The invention is therefore based on the problem of providing a pelotte that, despite its beneficial massage function, reduces slippage of the pelotte during movement.

The invention is therefore also based on the problem of providing a pelotte which, despite its advantageous massage function and effect, is less bulky and ensures a high level of wearing comfort even during prolonged use.

The present invention solves the underlying technical problem by means of a pelotte according to claim 1.

The present invention solves the underlying technical problem by means of a pelotte comprising a pelotte base body, wherein the pelotte base body has a front side and a back side, wherein the front side of the pelotte base body has at least one protrusion, characterized in that the pelotte base body has at least two expansion areas arranged in pairs, wherein the two expansion areas divide the pelotte base body into a first pelotte base body area and a second pelotte base body area and wherein the at least two expansion areas are each at least one groove or at least one incision in the pelotte base body.

The at least one groove is therefore an expansion groove which advantageously allows expansion in the expansion area of the pelotte base body, in particular between the first pelotte base body area and the second pelotte base body area.

The paired arrangement of the expansion areas advantageously allows uniform expansion. In the paired arrangement, the first expansion area is preferably positioned at one edge area of the pelotte base body and the second expansion area is positioned at the opposite edge area of the pelotte base body. The paired arrangement is particularly advantageous if the pelotte base body has a cavity. Such cavities are present, for example, in patella pelottes or elbow pelottes. These cavities are generally known to those skilled in the art and serve to accommodate the patella or elbow so that the pelotte base body surrounds the patella or elbow.

In a preferred embodiment, the pelotte base body has a cavity.

In a preferred embodiment, the pelotte base body has at least two expansion areas which are separated in the pelotte base body by the cavity. Preferably, the two pairs of expansion areas are located next to the cavity, for example to the left and right of the cavity or above and below the cavity. Preferably, the cavity has a diameter of at least 3 cm, i.e., it is in particular a cavity that can enclose the patella, the elbow, i.e., the head of the ulna or the head of the ulna or radius at the wrist. The size and shape of such cavities are known to those skilled in the art. Such cavities usually frame bony or cartilaginous protrusions on the body (protrusions), so that the size of the cavities results from this.

A pelotte, in particular a patella pelotte, elbow pelotte or wrist pelotte with exactly two expansion areas is preferred.

In a particularly preferred embodiment, the at least one groove is located on the front side of the pelotte base body. In a particularly preferred embodiment, the pelotte base body has an outer face and an inner face facing the cavity, wherein the at least one groove begins on the outer face and does not extend to the inner face or begins on the inner face and does not extend to the outer face.

Thus, the groove, i.e. the expansion groove, is preferably not a continuous channel with two lateral openings, but rather an incision that has only one lateral opening or no lateral opening.

Preferably, the at least one groove of an expansion area, i.e. the expansion groove, has only one lateral opening or no lateral opening. Preferably, the at least one groove of an expansion area, i.e. expansion groove, has only one lateral opening.

Preferably, an expansion area consists of at least one groove, for example one groove, two grooves, three grooves, four grooves, five grooves or more grooves.

Preferably, the two expansion areas arranged in pairs have the same number of grooves and/or grooves of the same shape and size.

The number, width, and depth of the grooves, i.e., expansion grooves, influence the expandability.

Preferably, the groove, i.e. expansion groove, has a width of at least 0.1 millimeter. Preferably, the groove, i.e. expansion groove, has a width of at least 0.3 millimeter. Preferably, the groove, i.e. expansion groove, has a width of 0.1 millimeter to 100 millimeter, in particular of 0.3 millimeter to 50 millimeter. It has been shown that this achieves the above-mentioned properties in a particularly advantageous manner. Preferably, the groove, i.e. the expansion groove, has a depth of 0.1 millimeter to 10 millimeter, in particular of 0.5 millimeter to 3 millimeter. It has been shown that this achieves the above-mentioned properties in a particularly advantageous manner.

According to a preferred further development of the invention, the groove, i.e. the expansion groove, has a constant depth or at least two different depths in its longitudinal extension.

According to a preferred embodiment of the invention, the groove, i.e., expansion groove, has a depth that corresponds to at least 10% of the height of the base body at the edge of the groove. The groove can be designed similarly to a “record groove.” Thus, it can be variable in width and depth.

In a preferred embodiment, the at least one protrusion is a pelotte pressure element. In a preferred embodiment, the at least one protrusion is nubs and/or ridges. Suitable protrusions are known to the skilled person. The pelotte may also have several protrusions, in particular different protrusions, which are positioned differently on the front side of the pelotte.

The protrusions may be made of the same material as the pelotte base body or of a different material. Preferably, the protrusions are made of a different material.

In a preferred embodiment, the protrusions are harder than the pelotte base body.

In this variant, the mobility and thus the massage function of the pelotte is also adjusted by the choice of materials. In particular, the base body and/or the protrusion, in particular the pelotte pressure element, are made of a viscoelastic material which allows advantageous deformation of the pelotte during use, leading to the desired massage effect. In particular, the materials are selected so that they differ from each other in terms of their rigidity or elasticity.

According to a preferred first embodiment, the protrusion, in particular the pelotte pressure element, is made of a material that is harder than the material of the base body. This allows the protrusions to transmit a higher pressure to the area to be treated at specific points. However, the mobility of the protrusions is reduced by the harder design.

According to a second embodiment of the invention, the protrusion, in particular the pelotte pressure element, is made of a material that is softer than the material of the base body, so that it has a higher elasticity and reduced stiffness compared to the base body. This gives the pressure projections a high degree of mobility, which in particular causes chaotic movement of in particular several pressure projections of the pelotte pressure element.

In a preferred embodiment, the material of the protrusion, in particular of the pelotte pressure element, has a Shore hardness of at least 40 Shore 00, preferably at least 6 Shore A and at most 20 Shore A. In a preferred embodiment, the material of the protrusion, in particular of the pelotte pressure element, has a Shore hardness of at least 20 Shore A and at most 80 Shore A. In a preferred embodiment, the material of the protrusion, in particular of the at least one pelotte pressure element, has a hardness of at least 20 Shore A and at most 60 Shore A. In a preferred embodiment, the material of the protrusion, in particular of the at least one pelotte pressure element, has a hardness of at least 20 Shore A, more preferably of at least 25 Shore A. In a preferred embodiment, the material of the protrusion, in particular of the at least one pelotte pressure element, has a hardness of at most 50 Shore A. In a preferred embodiment, the material of the protrusion, in particular of the at least one pelotte pressure element, has a hardness of at most 49 Shore A, more preferably of at most 45 Shore A. Preferably, the material of the protrusion, in particular of the at least one pelotte pressure element, has a hardness of at least 35 to at most 45 Shore A. Preferably, the material of the protrusion, in particular of the at least one pelotte pressure element, has a hardness of approximately 40 Shore A.

In a preferred embodiment, the material of the pelotte base body has a Shore hardness of at least 20 Shore 00 and at most 60 Shore 00 and/or the material of the protrusion, in particular of the pelotte pressure element, has a Shore hardness of at least 40 Shore 00, preferably at least 6 Shore A and at most 20 Shore A.

Preferably, the at least one protrusion is designed in the form of a knob, a cone, a cylinder, a pyramid, a cone, and/or a mushroom. Depending on the intended application, a different shape is suitable here, and if there are several protrusions, these may also have different shapes. The protrusions and the design of the pelotte according to the invention ensure that the movable protrusions exert an alternating pressure, such as that exerted by a therapist during a massage, so that the pressure effect on sensitive nerves, lymph and blood vessels is maintained for a long time. This is particularly advantageous if the pelotte is designed as a knee pelotte and a corresponding massage effect is applied to the base of the meniscus and the Hoffa's fat pad.

Preferably, one or at least one of the multiple protrusions is aligned in its longitudinal extension perpendicular to the outer surface of the pelotte base body or inclined towards the outer surface of the base body. By inclining the protrusion towards the outer surface, the protrusion is already given a direction of deformation during treatment. If the protrusion is loaded against the inclination, it acts particularly stiff and hard; if it is loaded in the direction of the inclination, it deflects more quickly or with less force. This allows the pressure distribution to be optimally adjusted to the respective patient when using the pelotte, depending on the application. If the protrusion extends perpendicular to the outer surface of the base body, it can be deformed equally or with equal force in all directions transverse to its longitudinal extension.

In a preferred embodiment, in particular as a patella pelotte, the pelotte base body has at least one, preferably two wings, wherein the at least one wing has at least one protrusion, preferably nubs and/or ridges.

The pelotte is preferably a knee pelotte, an ankle pelotte, a back pelotte, an elbow pelotte, a pelotte for the arm area or a pelotte for the abdominal area.

The pelotte according to the invention is preferably a pelotte for the kneecap, a pelotte for the elbow or a pelotte for the wrist. Preferably, the pelotte according to the invention is a pelotte for the kneecap, i.e. a patella pelotte.

In a preferred embodiment, the pelotte base body has a recess in the front side for the at least one protrusion, in particular the at least one pelotte pressure element, the contour of which corresponds to the contour of the at least one protrusion, preferably of the pelotte pressure element, at least substantially such that a groove is formed between the base body and the protrusion, preferably the pelotte pressure element, at least in sections in the circumferential direction of the protrusion, preferably of the pelotte pressure element. This groove is a spacing groove, i.e. not the expansion groove. This spacing groove is therefore an additional groove to the at least one expansion groove. This has the advantage that, by means of a structurally simple adjustment, the mobility of the protrusion or protrusions is increased and thus the massage effect remains at least the same or is improved in the case of softer, harder and/or smaller protrusions. The groove defines a lateral distance between the base body and the pelotte pressure element. This means that the pelotte pressure element and, in particular, the protrusion arranged thereon are not in direct lateral contact with the base body. The at least one pelotte pressure element is thus mounted in the base body in a floating manner. As a result, during use and under load, the pressure projection is supported less strongly by the base body than before and is therefore more easily deformable. This allows the pelotte to be smaller in height, i.e. in relation to the height or length of the pressure projections, while maintaining the same massage effect. In particular, it is intended that the respective pressure projection is flush with the base element at the side, i.e. lies directly on the outer edge of the base element which defines the outer contour of the pelotte pressure element. The groove thus interacts directly with the respective pelotte pressure element and prevents the respective pelotte pressure element from being supported laterally directly by the material of the base body.

According to a preferred embodiment of the invention, the groove extends over the entire circumference of the protrusion, in particular of the pelotte pressure element. This ensures that, regardless of the design of the protrusion, it can move in the advantageous manner described above. Due to the formation of a circumferential groove, the contour of the recess corresponds to the contour of the pelotte pressure element or the base element, which also simplifies the manufacture of the pelotte.

Preferably, the spacing groove has a width, i.e., a distance from the base element to the base body, of 0.3 millimeter. It has been shown that this achieves the above-mentioned properties in a particularly advantageous manner.

According to a preferred further development of the invention, the groove, i.e. the spacing groove, has a constant depth or at least two different depths in its longitudinal extension. The depth of the groove changes or influences the height range in which the base body and the pelotte pressure element are spaced apart from each other. As the depth of the groove decreases, the base body has a greater supporting effect on the base element and the respective pressure projection. As the depth increases, the influence and thus the stiffening of the pelotte pressure element decreases. Thus, the target function of the pelotte pressure element can be adjusted or adapted by both the width and the depth of the groove, viewed in the circumferential direction of the base body.

According to a preferred embodiment of the invention, the groove, i.e. the spacing groove, has a depth that corresponds to at least 10% of the height of the base body at the edge of the groove. The groove can be designed similarly to a “record groove.” This means that its width and depth can be

According to a preferred embodiment of the invention, the pelotte pressure element and the base body are made of the same material. This results in the advantageous mobility of the pressure projections due to the spacing groove. However, the use of the same material reduces the manufacturing effort. Preferably, the base body and the base element are then also formed in one piece with each other, so that a single-component body is obtained. This ensures that the pelotte can be manufactured in a particularly time-and cost-saving manner. The mobility of the pressure projections or the pelotte pressure element is then individually adapted to the requirements by selecting a suitable groove width and/or groove depth for the spacing groove.

The present invention also relates to an orthopedic aid, for example a bandage or orthosis, comprising a pelotte according to the invention. The present invention also relates in particular to a bandage or orthosis comprising a pelotte according to the invention, for example a knee bandage or orthosis, an ankle bandage or orthosis, a back bandage or orthosis, an elbow bandage or orthosis, a bandage or orthosis for the arm area or a bandage or orthosis for the abdominal area.

Preferably, it is a knee joint bandage or orthosis, an elbow bandage or orthosis, or a wrist bandage or orthosis. Preferably, it is a knee joint bandage or orthosis. Preferably, it is a knee joint bandage.

The orthosis or bandage according to the invention is characterized by the pelotte according to the invention. This results in the advantages already mentioned above. Preferably, the orthosis is an elastic knitted orthosis or the bandage is an elastic knitted bandage, in particular a knee orthosis or knee bandage.

Further advantages and preferred features and combinations of features result from the above description and from the claims. The invention will now be explained in more detail with reference to the drawings. For this purpose,

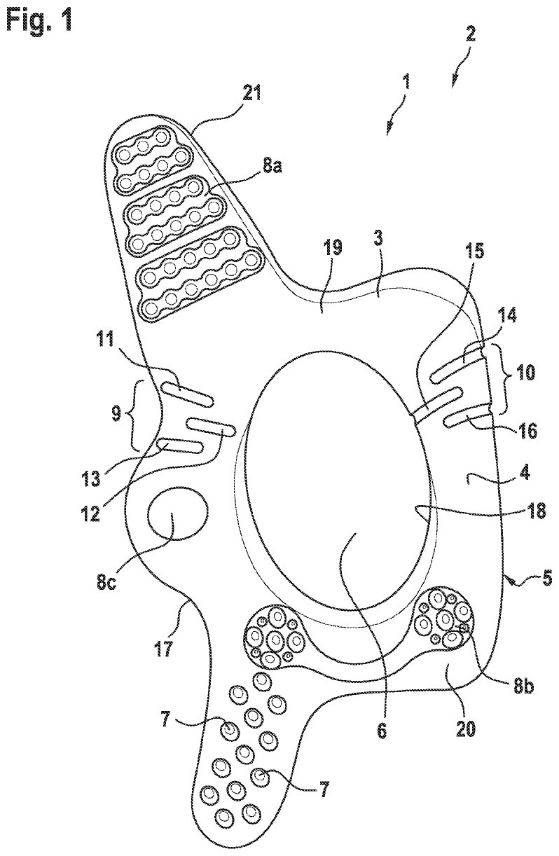

FIG. 1 shows an advantageous pelotte in a perspective top view,

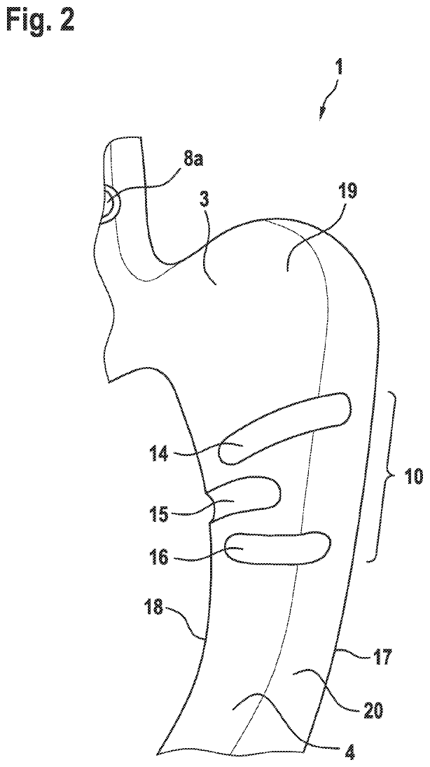

FIG. 2 shows a section of an expansion area of the pelotte from FIG. 1,

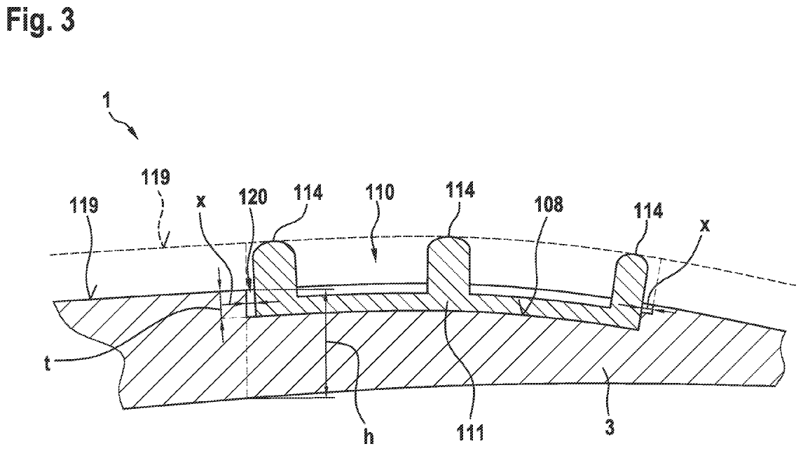

FIG. 3 shows a section of a preferred embodiment of a pelotte according to the invention.

FIG. 1 shows an advantageous pelotte 1 for an orthosis or bandage 2 not shown in detail here. The embodiment of the pelotte shown is a patella pelotte. The pelotte 1 has a pelotte base body 3, which is essentially flat and made of an elastic, in particular viscoelastic material. The base body 3 has a three-dimensional outer contour with a front side 4 as the treatment side and a back side 5 facing away from the front side 4. According to the present embodiment, the outer contour of the base body 3 is characterized by several wings which protrude from a base ring, in particular radially outward, wherein an oval-shaped opening is also formed in the base ring as a cavity 6. The pelotte has several protrusions, i.e. pelotte pressure elements of various shapes, on the wings and on the base ring, wherein some pelotte pressure elements 7 are made of the same material as the base body and some pelotte pressure elements 8a, 8b, 8c are made of a harder material.

According to the invention, the pelotte has two pairs of expansion areas 9, 10, each of which is formed by three grooves 11, 12, 13, 14, 15, 16 cut into the base body 3, which act as expansion grooves. None of the expansion grooves 11, 12, 13, 14, 15, 16 extend from the outer face 17 of the pelotte base body 3 to the inner face 18 of the pelotte base body facing the cavity 6. The pair of expansion areas 9, 10 divide the pelotte base body 3 into a first pelotte base body area 19 and a second pelotte base body area 20, wherein, in the case of the patella pelotte, the first pelotte base body area 19 is positioned at the top and the second pelotte base body area 20 is positioned at the bottom.

The paired expansion areas 9, 10 allow the upper, first pelotte base body area 19 and the lower, second pelotte base body area 20 to move away from each other much more when the knee is bent than would be the case with a pelotte without the expansion areas. On a clock, the positions of the expansion areas 9, 10 can be described as “at 2 o'clock and 10 o'clock.” However, other positions are also possible, for example, “at 3 o'clock and 9 o'clock” or “at 4 o'clock and 8 o'clock.” An essential feature, however, is that all expansion areas only perform the desired function when used in pairs.

Alternatively, this type of stretchability could only be achieved by using a softer base material for the pelotte itself. This would mean that the functions of the pelotte, i.e. positional stability of the kneecap and the entire knee brace, spacer for the elastic knit spanning the kneecap, would no longer be possible to the required extent. Ideally, the expansion areas, in particular the expansion grooves, are located to the right and left of the joint gap of the knee joint. This is because when the knee is bent, the upper part 19 of the pelotte must be able to maintain its position relative to the upper end of the kneecap. Only then is it ensured that protrusions, in particular pelotte pressure elements 8a, located on the upper area of the pelotte 1 maintain their position. In pelottes of the prior art, when the nubbed area above the pelotte oval is used, it is often displaced so much that the nubs located on it “bend” instead of applying massage pressure perpendicular to the base body surface as intended. However, this perpendicular effect of the nubs is advantageous as a targeted imitation of therapeutic massages on the main muscle, the vastus medialis. However, if the nubs located in this area tilt away, the sensorimotor response of the muscle to the stimulus is not as required. The paired expansion areas 9, 10 according to the invention advantageously stabilize the position of the two pelotte base body areas.

In the patella pelotte 1 shown, a lateral wing 21 provided with pelotte pressure elements 8a is molded above the main muscle “vastus medialis,” which, when pressure is applied to the muscle, produces a massage with release of the contractures and a sensorimotor response of the muscle.

The combination of the expansion areas 9, 10 according to the invention with the intermittently massaging pelotte pressure elements 8a incorporated above the vastus medialis muscle produces the desired pain relief by relaxing the painfully contracted muscle in arthrosis patients, in particular by positively influencing the trigger points. This corresponds to the pain-relieving effect produced by the physiotherapist with a manual friction massage. Further pain-relieving advantages are the reduced tensile stress on the patellar ligament, the reduced shear stress on the arthritic patella and the reduced risk of the bandages slipping.

The surprising advantages of a combination of expansion areas 9, 10 and pelotte pressure elements 8a can be transferred by specialists to other pelottes such as elbow pelottes or wrist pelottes.

To illustrate the expansion areas, FIG. 2 shows an enlarged section of the pelotte 1. The expansion area 10 consists of the three expansion grooves 14, 15, 16, which are provided on the front side 4 of the pelotte base body 3. The expansion grooves are all discontinuous, and therefore do not form a channel between the inner face 18 of the pelotte base body 3 facing the cavity and the outer face 17 of the pelotte base body 3, but rather the grooves 14 and 16 are only open towards the outer face 17 of the pelotte base body 3 and the groove 15 is only open towards the inner face 18 of the pelotte base body 3 facing the cavity. 3. The expansion area 10 divides the pelotte base body 3 into two pelotte base body areas 19, 20. The upper, first pelotte base body area 19 has the pelotte pressure elements 8a, which remain better positioned during movement due to the paired expansion areas 10, 11.

To clarify the pelotte pressure elements with spacing grooves, FIG. 3 shows a longitudinal sectional view of the pelotte 1 in the area of the pelotte pressure element 8b, here designated 110, according to FIG. 1.

The recess 108 thus has a depth which, according to the present embodiment, is at least as great as the height of the base element 111 of the pelotte pressure element 110. The pressure projections 114 are designed to be so high that they protrude from the recess of the base body 3 of the pelotte 1. While, according to the present embodiment, the recess 108 has a straight or flat bottom, according to a further embodiment-not shown here-it is provided that the bottom of the recess deviates from a straight plane. This allows the behavior of the pressure projections 114 to be further influenced during use and, for example, a preferred bending direction to be specified.

The contour or inner contour of the respective recess 108 corresponds at least substantially to the contour or outer contour of the respective pelotte pressure element 110, in particular that of the respective base element 111. The contour of the respective recess 108 is selected such that, at least in sections, a lateral distance x remains in the circumferential direction of the respective base element 111. The distance x creates a groove, i.e. a spacing groove 120, between the respective pelotte pressure element 110 and the base body 3. This spacing groove 120 extends at least in sections along the circumference of the respective pelotte pressure element. The distance x formed by the groove 120 is preferably 0.1 mm to 3 m, preferably 0.5 mm to 1.5 mm.

According to an advantageous embodiment, the spacing groove 120 extends over the entire circumference of the pelotte pressure element 110. In its longitudinal extension, the groove 120 preferably has a constant depth t.

The groove 120 preferably has a depth t which corresponds to at least 10% of the height h of the base body 3 at the edge of the groove 120.

The spacing groove 120 has the effect of advantageously influencing the deflection behavior and also a deflection direction of the pressure projection 114.

The pressure projections 114, which are designed in the form of nubs with rounded ends, are preferably made of a harder material than the base body 3. In the case of pelottes whose pressure projections 114 are harder than the base body 3, the massage effect that can be achieved by the pelotte 1 or an orthosis or bandage 2 having a pelotte 1 is higher.

The depth t and width x of the groove 120 surrounding the respective pressure projection or the base element 111 determines the mobility of the respective associated pressure projection 114. Because the material of the base body 3 immediately adjacent to the respective pressure projection 114 is thinner or removed, the individual pressure projection 114 can flex better due to the distance x resulting from the groove 120 and also move better in general within the extended edge area. This applies regardless of whether the respective pressure projection 114 is completely sunk into the recess 108 and, for example, is flush with the outer surface 119 of the base body 3, or whether the respective pressure projection 114 protrudes from the outer surface 119 as shown in the present embodiments.

The groove 120 helps to make the pelotte pressure element 110 smaller overall than would be the case with flush mounting on a flat mounting surface without a groove. The pelotte pressure elements 110 are thus mounted in a floating manner in the area of the groove 120.

For certain applications, it may be advantageous if the pressure projections or the pelotte pressure elements 110 do not protrude or only protrude slightly from the base body 3, but are very movable at their respective positions, in particular with regard to their pressure projections 114. According to a further embodiment, the pressure projections 114 thus end in particular flush with the outer surface 119, as shown in FIG. 3 by way of example by a dashed line of the outer surface.

### LIST OF REFERENCE NUMBERS

1 pelotte
2 orthosis or bandage
3 pelotte base body
4 front side
5 back side
6 cavity
7 protrusions/pelotte pressure elements
8a to c pelotte pressure elements
9 first expansion area
10 second expansion area
11 expansion groove
12 expansion groove
13 expansion groove
14 expansion groove
15 expansion groove
16 expansion groove
17 outer face of the pelotte base body
18 inner face of the pelotte base body facing the cavity
19 first pelotte base body area
20 second pelotte base body area
21 upper pelotte wing
108 recess
110 pelotte pressure elements
111 base element of the pelotte pressure element
114 pressure projections
119 outer surface
120 spacing groove
h height of the base body
t depth
x distance

1. A pelotte comprising a pelotte base body, wherein the pelotte base body has a front side and a back side, wherein the front side of the pelotte base body has at least one protrusion, characterized in that the pelotte base body has at least two expansion areas arranged in pairs, wherein the two expansion areas divide the pelotte base body into a first pelotte base body area and a second pelotte base body area and wherein the at least two expansion areas are each at least one groove in the pelotte base body, wherein the pelotte base body has a cavity, characterized in that the pelotte base body has an outer face and an inner face directed toward the cavity, wherein the at least one groove begins on the outer face and does not extend to the inner face or begins on the inner face and does not extend to the outer face.

2. (canceled)

3. The pelotte according to claim 1, wherein the pelotte base body has at least two expansion areas which are separated in the pelotte base body by the cavity.

4. The pelotte according to claim 3 with exactly two expansion areas.

5. The pelotte according to claim 1, wherein the at least one groove is located on the front side of the pelotte base body.

6. The pelotte according to claim 1, wherein the cavity has a diameter of at least 3 cm.

7. The pelotte according to claim 1, wherein the at least one protrusion is nubs and/or ridges.

8. The pelotte according to claim 1, wherein the protrusions are harder than the pelotte base body.

9. The pelotte according to claim 1, wherein the protrusions are softer than the pelotte base body.

10. The pelotte according to claim 1, wherein the pelotte base body has at least one, preferably two wings, wherein the at least one wing has at least one protrusion, preferably nubs and/or ridges.

11. The pelotte according to claim 1, wherein it is a pelotte for the kneecap, a pelotte for the elbow or a pelotte for the wrist.

12. A bandage or orthosis comprising a pelotte according to claim 1.

13. The bandage or orthosis according to claim 12, which is a knee joint bandage or orthosis, an elbow bandage or orthosis, or a wrist bandage or orthosis.
